(19) United States
(12) Plant Patent Application Publication
(10) Pub. No.: US 2012/0131710 P1
(43) Pub. Date: May 24, 2012

(54) Pearlbush shrub named 'Blizzard'

(50) ;

(75) Inventor: Thomas G. Ranney, Arden, NC (US)

(73) Assignee: North Carolina State University, Raleigh, NC (US)

(21) Appl. No.: 12/927,738

(22) Filed: Nov. 22, 2010

(51) Int. Cl. A01H 5/00

(52) U.S. Cl. PLT/226

(57) ABSTRACT

‘Blizzard’ is a new pearlbush plant particularly distinguished by its compact habit, smaller plant height and width, larger flower diameter, and larger genome size.

### GENUS AND SPECIES

Exochorda sp.

### VARIETY DENOMINATION

‘Blizzard’

### BACKGROUND OF THE NEW PLANT

The present invention comprises a new and distinct variety of Exochorda, botanically known as Exochorda sp., and hereinafter referred to by the variety name ‘Blizzard’. This new pearlbush shrub was developed through a breeding program in Mills River, N.C. ‘Blizzard’ is an F₂ hybrid between the female parent, Exochorda serratifolia (unpatented) and the male parent, Exochorda×macrantha ‘The Bride’ (unpatented). The first asexual propagation of ‘Blizzard’ was carried out in the summer of 2003 by rooting stem cuttings in Mills River, N.C. and has been asexually reproduced repeatedly by vegetative cuttings in North Carolina over a 7 year period.

‘Blizzard’ roots readily from softwood cuttings from juvenile plants treated with a basal dip of 5,000 ppm indole butyric acid (potassium salt) in water. ‘Blizzard’ has been found to retain its distinctive characteristics through successive asexual propagations via vegetative cuttings.

Plant Breeder's Rights for this variety have not been applied for. ‘Blizzard’ has not been made publicly available or sold more than one year prior to the filing date of this application.

### SUMMARY OF THE INVENTION

The following are the most outstanding and distinguishing characteristics of this new variety when grown under normal horticultural practices at in Mills River, N.C.

1. A compact shrub habit;

2. Upright to rounded form;

3. Extremely floriferous with large flowers; and

4. A polyploid cytotype with a relative 2C genome size of approximately 2.2 pg.

### DESCRIPTION OF THE PHOTOGRAPHS

This new pearlbush variety is illustrated by the accompanying photographs which show the plant's form, foliage and inflorescences. The colors shown are as true as can be reasonably obtained by conventional photographic procedures. The photographs were taken in Mills River, N.C.

FIG. 1 shows a 6 year old, field-grown plant in full bloom in April, 2008.

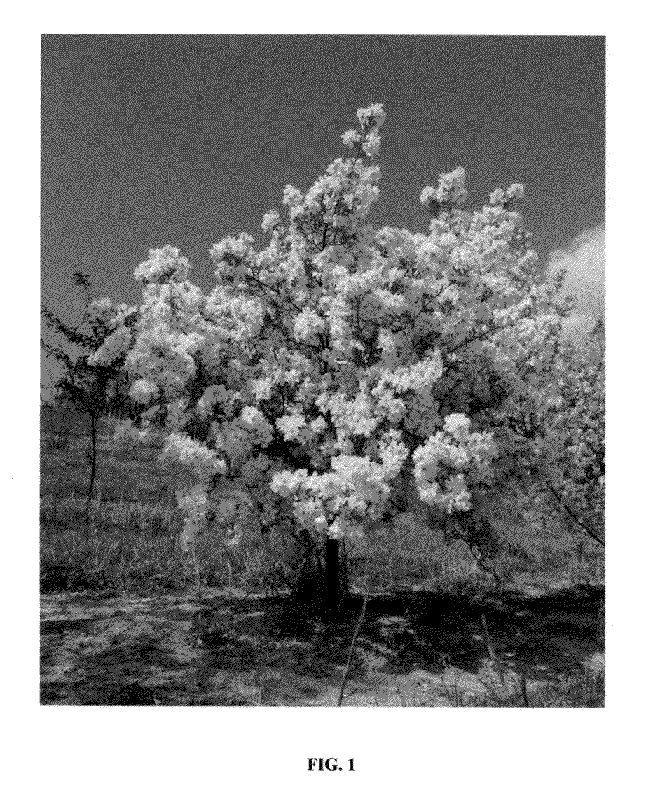

FIG. 2 shows the flowers of a greenhouse-grown plant in April, 2009.

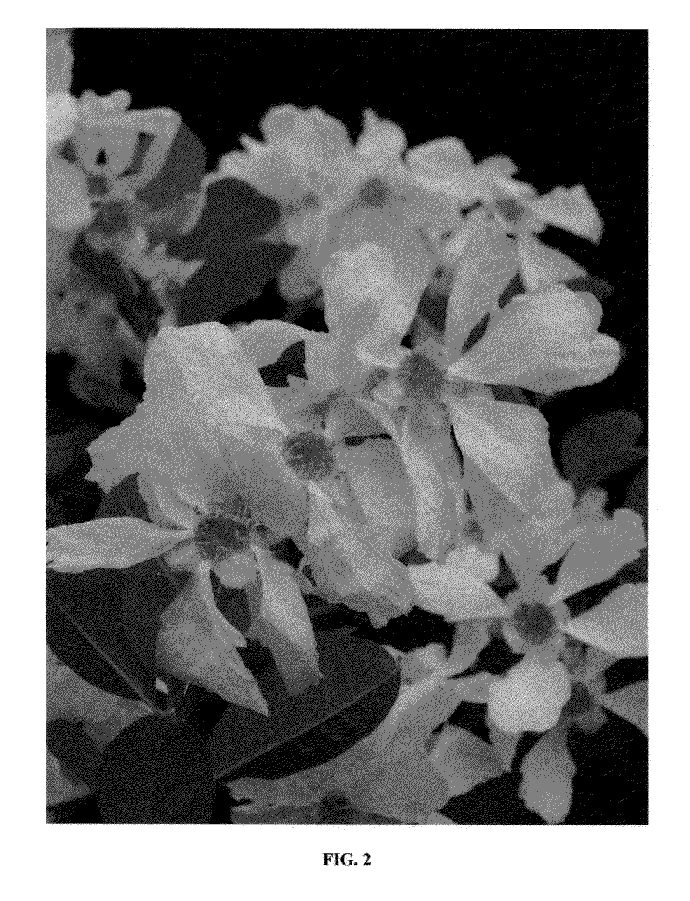

### DESCRIPTION OF THE NEW VARIETY

The following detailed description sets forth the distinctive characteristics of ‘Blizzard’. The detailed description was taken on 6-year-old plants growing under landscape conditions in Mills River, N.C. in April 2008. Color references are to the R.H.S. Colour Chart of The Royal Horticultural Society of London (R.H.S.), 2001 Edition.

### DETAILED BOTANICAL DESCRIPTION OF THE PLANT

Classification:
   Botanical name.—Exochorda sp.
   Common name.—Pearlbush.
Parentage: An F₂ hybrid between the following parents.
   Female parent.—Exochorda serratifolia (unpatented).
   Male parent.—Exochorda×macrantha ‘The Bride’ (unpatented).
Plant description:
   Growth habit.—Compact.
   Plant shape.—Upright when young, eventually becoming rounded with age.
   Height at maturity.—1.2 meters to 1.5 meters.
   Width at maturity.—1.2 meters to 1.5 meters.
Stem:
   Mature stem color.—RHS N200A (Brown).
   Immature stem color.—RHS 143A (Yellow-green).
Leaves:
   Type.—Deciduous.
   Arrangement.—Alternate.
   Shape.—Obovate to obelliptic.
   Apex.—Obtuse mucronulate.
   Base.—Cuneate.
   Margin.—Entire but occasionally serrate towards the apex.
   Mature leaf size.—Length: Average is 5.12 cm (range is 4.8 cm to 5.6 cm) Width: Average is 2.92 cm (range is 2.6 cm to 3.3 cm).
   Immature leaf color.—Adaxial (upper) surface: Flushes RHS 143A (Green), which changes to RHS 137A (Green) Abaxial (lower) surface: RHS 144A (Yellow-green) which changes to RHS 137A (Green).
   Mature leaf color.—Adaxial (upper) surface: RHS 137B (Green) Abaxial (lower) surface: RHS 138B (Green).
   Mature leaf texture.—Adaxial (upper) surface: Glabrous Abaxial (lower) surface: Pubescent.
   Leaf attachment.—Petiolate.
   Petiole.—Shape: Moderately sulcate Length: Average is 0.55 cm (range is 0.47 cm to 0.62 cm) Width: Average is 0.096 cm (range is 0.073 cm to 0.14 cm) Surface: Glabrous (not pubescent).
Inflorescence:
   Inflorescence type.—Raceme with the occasional panicle.
   Number of flowers per inflorescence.—4 to 10.
   Flower diameter.—5.7 cm (range is 5.0 to 6.4 cm).
   Type.—Perfect, simple.
   Flowering season.—Early spring through late spring; mid-April in Mills River, N.C.
   Lastingness of flower on the plant.—7 to 21 days.
   Fragrance.—Absent.
   Length at anthesis.—Average is 6.0 cm (range is 3.6 cm to 8.6 cm).
   Self cleaning or persistent.—Self-cleaning.
   Petal.—Number of petals per flower: Average is 5 (range is 5 to 6) Color, adaxial (upper) surface at anthesis: RHS 155C (White) Color, abaxial (lower) surfaces at anthesis: RHS 155C (White) Surface texture and appearance: Glabrous (not pubescent), soft, thin and lustrous (slightly shiny) Shape: Obovate Apex: Obtuse Base: Attenuate Margin: Irregularly crenulate Length: Average is 2.36 cm (range is 2.13 cm to 2.67 cm) Width: Average is 1.29 cm (range is 0.93 cm to 1.44 cm) Petals, fused or unfused: Unfused.
   Pedicel.—Shape: Cylindrical, laterally compressed Length: Average is 0.37 cm (range is 0.15 cm to 0.81 cm) Width: Average is 0.086 cm (range is 0.06 cm to 0.11 cm) Color: RHS 143A (Green) Surface texture: Glabrous (not pubescent).
   Peduncle.—Shape: Cylindrical, laterally compressed Length: Average is 1.19 cm from last flower to first pedicel (range is 0.33 cm to 1.72 cm) Width: Average is 0.17 cm at base (range is 0.14 cm to 0.20 cm) Color: Closest to RHS 143A (Green) Surface texture: Glabrous (not pubescent).
Reproductive organs:
   Stamen.—Quantity per flower: Average is 31 (range is 28 to 36) Shape: Filament Color: Translucent RHS 155C (White) Attachment: Dorsifixed Anther: Length: Average is 0.11 cm (range is 0.082 cm to 0.137 cm) Width: Average is 0.063 cm (range is 0.044 cm to 0.071 cm) Shape: Ovoid Color: RHS 11B (Yellow) Pollen color: RHS 11B (Yellow) Pollen amount: Moderate to copious, similar to parental lines.
   Pistil.—Generally abortive.
   Ovary.—Generally abortive or lacking.
Fruit:
   General.—Fruit are infrequent; when present, a coccetum with 5 fused carples.
   Shape.—Flattened sphere.
   Color.—Initially RHS 139D (Green) eventually turning 177D (Brown).
   Length.—1.2 cm.
   Width.—1.5 cm.
Seed: No viable seed have been observed.
Disease and insect resistance: No significant disease or insect pests have been observed.
Genome size: The large flowers size of ‘Blizzard’ indicated that it may be a polyploid. Flow cytometry was conducted to determine DNA content which is directly correlated with ploidy level among closely related taxa. Holoploid, 2C DNA content was determined on newly expanded leaf tissue. Nuclei were extracted, stained with 4′, 6-Diamidino-2-phenylindole (DAPI), and analyzed (minimum of 5,000 nuclei per sample) using a flow cytometer (PA-I, Partec, Münster, Germany). Pisum sativum L. ‘Ctirad’, with a known genome size of 8.75 pg was used as an internal standard. Mean (n=2), 2C DNA content for ‘Blizzard’ was 2.2±0.003 (SEM) pg, approximately twice the DNA content of either parent, indicating that ‘Blizzard’ is a polyploidy cytotype (see Table 1).

### COMPARISON WITH PARENTAL AND COMMERCIAL VARIETIES

‘Blizzard’ is distinguished from the female parent, Exochorda serratifolia (unpatented) in that ‘Blizzard’ has a compact habit, has a smaller mature plant height and width, has a larger flower diameter, and a larger 2C genome size than the female parent, Exochorda serratifolia.

‘Blizzard’ is distinguished from the male parent Exochorda×macrantha ‘The Bride’ (unpatented) in that ‘Blizzard’ has an upright to rounded habit, has a larger flower diameter, and larger 2C genome size than the male parent ‘The Bride’.

Table 1 shows the specific differences between ‘Blizzard’ and Exochorda serratifolia (unpatented) and Exochorda×macrantha ‘The Bride’ (unpatented).

TABLE 1
Additional comparisons of ‘Blizzard’ with parental lines

| | Taxa | | |
|---|---|---|---|
| | | | E. × macrantha |
| Trait | ‘Blizzard’ | E. serratifolia | ‘The Bride’ |
| Flower diameter | 5.0 cm to 6.4 cm | 2.8 cm to 4.5 cm | 3.1 cm to 3.7 cm |
| Petal number per | 5 to 6 | 5 to 11 | 5 or 6 |
| flower | | | |
| Pistil | Abortive | Fasciated ovary | Fasciated ovary |
| Plant habit | Compact shrub | Large shrub/ | Open shrub |
| | | small tree | |
| Plant shape | Upright/rounded | Open/spreading | Arching |
| Plant height | 1.2 m - 1.5 m | 2.5 m - 3.7 m | 1.5 m - 2.5 m |
| Plant width | 1.2 m - 1.5 m | 2.4 m - 3.7 m | 1.2 m - 2.4 m |
| Relative Genome | 2.2 ± 0.003 pg | 1.1 ± 0.009 pg | 1.2 ± 0.005 pg |
| Size (2C DNA | | | |
| content ± SEM) | | | |

When ‘Blizzard’ is compared to the commercial variety E. serratifolia ‘Northern Pearls’, ‘Blizzard’ is more compact with a upright/rounded form reaching approximately 1.5 m high, while ‘Northern Pearls’ has an arching vase shape and reaches 2.4 m.

I claim:

1. A new and distinct variety of pearlbush shrub as described and shown herein.
